(19) United States
(12) Patent Application Publication
(10) Pub. No.: US 2025/0000868 A1
(43) Pub. Date: Jan. 2, 2025

(54) USE OF ADENOSINE A2B RECEPTOR AS TARGET IN DRUGS FOR PREVENTION AND/OR TREATMENT OF PRURITUS

(71) Applicant: The Fourth Affiliated Hospital, ZJU, Jinhua City (CN)

(72) Inventors: Xiangyao LI, Jinhua City (CN); Yanna LIAN, Jinhua City (CN); Li LIU, Jinhua City (CN); Cheng WU, Jinhua City (CN)

(73) Assignee: The Fourth Affiliated Hospital, ZJU, Jinhua City, ZJ (CN)

(21) Appl. No.: 18/446,558

(22) Filed: Aug. 9, 2023

(30) Foreign Application Priority Data
Jun. 29, 2023 (CN) 2023107869248

(51) Int. Cl. A61K 31/522; A61P 17/04

(52) U.S. Cl. CPC A61K 31/522; A61P 17/04

(57) ABSTRACT

The present disclosure provides the use of an adenosine A2B receptor as a target in drugs for prevention and/or treatment of pruritus and belongs to the technical field of biological pharmaceuticals. Expression of genes encoding major ligand-gated ion channels is detected based on previously reported single-cell RNA sequencing data of mouse dorsal root ganglions (DRGs), and Adora2b is detected to be highly expressed in Neuron_10, suggesting that Adora2b and Adora2b receptor (the adenosine A2B receptor) may be involved in pruritus regulation. Experimental results show that histamine-induced mouse scratching behavior can be effectively prevented by blocking adenosine A2B receptor activity, indicating that the adenosine A2B receptor is involved in histamine-induced pruritus. The present disclosure further provides the use of a reagent for inhibiting biological functions of an adenosine A2B receptor in preparing drugs for the prevention and/or treatment of pruritus, providing a new means for the prevention and treatment of pruritus.

### CROSS REFERENCE TO RELATED APPLICATION

This patent application claims the benefit and priority of Chinese Patent Application No. 2023107869248 filed with the China National Intellectual Property Administration on Jun. 29, 2023, the disclosure of which is incorporated by reference herein in its entirety as part of the present application.

### TECHNICAL FIELD

The present disclosure belongs to the technical field of biological pharmaceuticals and particularly relates to the use of an adenosine A2B receptor as a target in drugs for prevention and/or treatment of pruritus.

### BACKGROUND

Pruritus is a chief complaint of patients who visit the dermatology clinic, and it is caused by inflammatory mediators, neurotransmitters, Neuropeptide and other destructive stimuli activate skin nerve terminals, leading to the transmission of pruritus signal from the peripheral skin through the spinal cord and the thalamus to the cerebral cortex. According to the duration of itch symptoms, pruritus can be divided into acute pruritus and chronic pruritus. The time course of the acute pruritus is shorter than 6 weeks, and that of the chronic pruritus is longer than 6 weeks. The prevalence rate of chronic pruritus is higher in the population. Due to continuous pruritus and extensive skin lesions, patients are often complicated with depression and insomnia, leading to a serious decrease in their quality of life.

Epidemiological investigation found that approximately 22%-90% of patients with major non-inflammatory diseases, like uremic pruritus, had concomitant itchy skin (Feramisco, et al., 2010). At present, clinically used antipruritics are still dominated by antihistamine drugs, but these drugs had little efficacy in most patients with chronic pruritus. Therefore, further interpretation of the production and regulation mechanism of itching sensation and an effective target thereof will provide an important theoretical instruction for research and development of new antipruritics. However, there has been no report of the drug target for pruritus yet.

### SUMMARY

In view of this, the present disclosure aims to provide the use of an adenosine A2B receptor as a target in drugs for prevention and/or treatment of pruritus.

The present disclosure provides use of an adenosine A2B receptor as a target in screening, developing or preparing drugs for prevention and/or treatment of pruritus.

Preferably, the pruritus includes at least one selected from the group consisting of uremic pruritus, chronic pruritus, and acute pruritus.

Preferably, the pruritus is histamine-or chloroquine-induced pruritus.

Preferably, the drugs includes a component for inhibiting adenosine A2B receptor activity.

IPreferably, the reagent for inhibiting biological functions of an adenosine A2B includes a monoclonal or polyclonal antibody of the adenosine A2B receptor, an antagonist of the adenosine A2B receptor, and a competitive ligand of the adenosine A2B receptor.

Preferably, the antagonist of the adenosine A2B receptor includes PSB-603.

Preferably, the PSB-603 is administered at a concentration of at least 10 μM.

Preferably, the pruritus includes at least one selected from the group consisting of uremic pruritus, chronic pruritus, and acute pruritus.

Preferably, the pruritus is histamine- or chloroquine-induced pruritus.

The present disclosure provides the use of an adenosine A2B receptor as a target in screening, developing or preparing drugs for prevention and/or treatment of pruritus. In the present disclosure, expression of genes encoding major ligand-gated ion channels is detected based on previously reported single-cell RNA sequencing data of mouse dorsal root ganglions (DRGs), and Gfra1 and Adora2b are detected to be highly expressed in Neuron_10, suggesting that Adora2b receptor, the adenosine A2B receptor, may be involved in pruritus regulation. Results of the scratching behavioral experiment show that histamine-induced mouse scratching behavior may be effectively presented by blocking adenosine A2B receptor activity. It is indicated that the adenosine A2B receptor is involved in histamine-induced pruritus. Therefore, the adenosine A2B receptor as a drug target is an effective way to treat pruritus.

### BRIEF DESCRIPTION OF THE DRAWINGS

FIGS. 1A-D illustrates the classification results of DRG neurons according to a gene expression profile, where FIG. 1A illustrates the Umap embedding projection of adult mouse DRG neuronal subtypes, where the number represents the number of Seurat clusters with a resolution of 0.4; FIG. 1B is a spot map showing an expression profile of the first three characteristic ferroptosis-related differentially expressed genes (fDEGs) in DRG neuronal subtypes, where spot size represents the percentage of positive cells, and color intensity represents the average expression of related genes; FIG. 1C is a spot map of cell type-labeled expression profile; and FIG. 1D is a summary based on the classification of previously reported neurons;

FIG. 2 is a spot map showing the expression of genes encoding adenosine triphosphate (ATP), adenosine, and cannabinoid receptors; note: spot size represents the percentage of positive cells, and color intensity represents the average expression of related genes; cluster analysis is conducted on cell types according to the percentage and average expression of related genes; a method of D2;

FIGS. 3A-C illustrate results of adenosine A2B receptor involvement in pruritus regulation, where FIG. 3A shows representative images of marks (arrows) of A2B-positive neurons and GFRα-1, with a scale of 20 μm; FIG. 3B shows the proportion of A2B-positive cells co-expressing GFRα-1 in DRGs (n=3 mice); and FIG. 3C illustrates dose-dependent inhibition of histamine-induced scratching behaviors by intrathecal injection of PSB-603 (histamine: His; PSB-603: P; n=7-8 mice in each group; data are expressed as mean ±SEM; *P<0.05, **P<0.01, ***P<0.001, one-way ANOVA, followed by Tukey's test for multiple comparisons); and

FIG. 4 illustrates the results of dose-dependent inhibition of chloroquine-induced scratching behaviors by PSB-603.

### DETAILED DESCRIPTION OF THE EMBODIMENTS

The present disclosure provides the use of an adenosine A2B receptor as a target in screening, developing or preparing drugs for prevention and/or treatment of pruritus.

The present disclosure has no particular limitation on the pruritus, as long as pruritus induced by inflammatory mediators, neurotransmitters, Neuropeptide and other destructive stimuli falls into the category of the pruritus related in the present disclosure, for example, uremic pruritus, chronic pruritus, and acute pruritus. In the present disclosure, the pruritus is preferably histamine- or chloroquine-induced pruritus. Experimental results show that epidermal injection of histamine or chloroquine can effectively induce mouse scratching behaviors.

In the present disclosure, the drugs preferably achieve the therapeutic purpose by inhibiting adenosine A2B receptor activity. Single-cell transcriptome data GSE155622 (Wang, K., Wang, S., Chen, Y., Wu, D., Hu, X., Lu, Y., et al. (2021). Single-cell transcriptomic analysis of somatosensory neurons uncovers temporal development of neuropathic pain. Cell Res. 31. doi: 10.1038/s41422-021-00479-9) and GSE154659 (Renthal, W., Tochitsky, I., Yang, L., Cheng, Y.-C., Li, E., Kawaguchi, R., et al. (2020a). Transcriptional Reprogramming of Distinct Peripheral Sensory Neuron Subtypes after Axonal Injury. Neuron 108, 128-144.e9. doi: 10.1016/j.neuron.2020.07.026) are downloaded from the Gene Expression Omnibus (GEO) database (http://www.ncbi.nlm.nih.gov/geo/). scRNA data (Speir, M. L., Bhaduri, A., Markov, N. S., Moreno, P., Nowakowski, T. J., Papatheodorou, I., et al. (2021). UCSC Cell Browser: Visualize Your Single-Cell Data. Bioinformatics 37, 4578-4580) are downloaded from UCSC website (https://cells.ucsc.edu/?ds=mouse-nervous-system+drg). Single-cell RNA sequencing data of mouse dorsal root ganglions (DRGs) are analyzed. It is found that Gfra1 and Adora2b are highly expressed in Neuron_10, suggesting that the Adora2b gene and an adenosine A2B receptor encoded thereby may be involved in pruritus regulation. On this basis, the present disclosure carries out immunohistochemistry (IHC) protocols. It is found that adenosine A2B receptor and GFRα-1 protein are co-expressed in DRG neurons. Scratching behavioral experiment verifies whether the adenosine A2B receptor is involved in regulation of scratching behaviors. It is indicated that adenosine A2B receptor activity is blocked by intrathecal injection of selective adenosine A2B receptor antagonist PSB-603, and that administration of PSB-603 effectively prevents histamine-induced scratching behaviors. It is indicated that the adenosine A2B receptor is an effective way to treat pruritus.

The present disclosure provides the use of a reagent for inhibiting biological functions of an adenosine A2B receptor in preparing drugs for the prevention and/or treatment of pruritus.

In the present disclosure, the reagent for inhibiting biological functions of an adenosine A2B preferably includes a monoclonal or polyclonal antibody of the adenosine A2B receptor, an antagonist of the adenosine A2B receptor, and a competitive ligand of the adenosine A2B receptor. In the examples of the present disclosure, the adenosine A2B receptor antagonist is used as a representative to illustrate efficacy in the prevention and treatment of the pruritus. The antagonist of the adenosine A2B receptor preferably includes PSB-603.

Experimental results show that alleviated animal scratching behaviors are related to a dosing concentration of the PSB-603, and the PSB-603 is preferably administered at a concentration of at least 10 μM. When 1 μM PSB-603 is administered, the number of animal scratches cannot be reduced.

The use of an adenosine A2B receptor as a target in drugs for prevention and/or treatment of pruritus provided by the present disclosure will be described in detail below with reference to examples. However, they should not be construed as limiting the protection scope of the present disclosure.

### Example 1

### Screening of a Gene for Pruritus Regulation

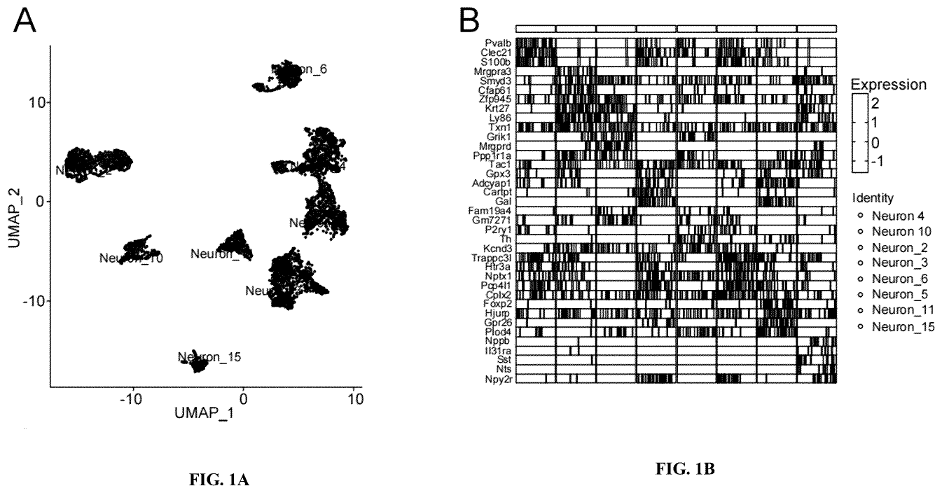

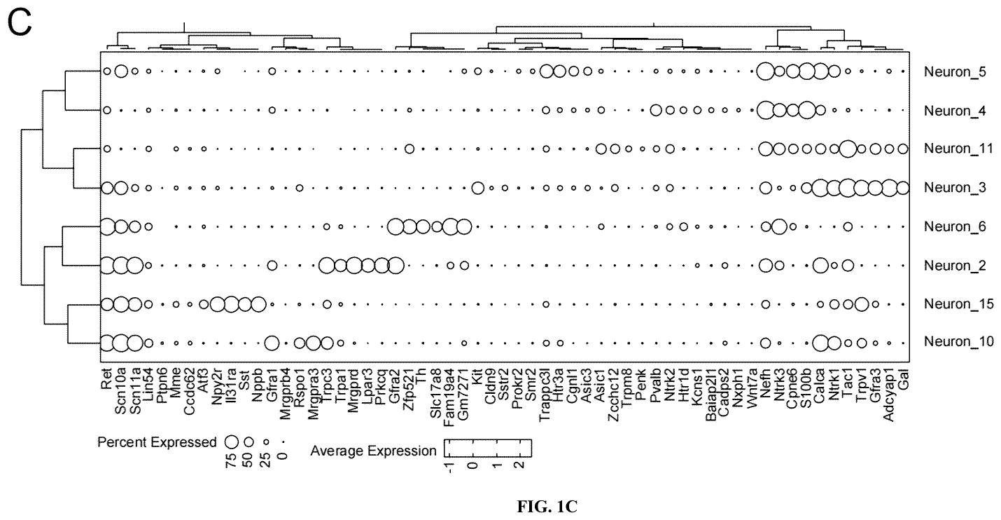

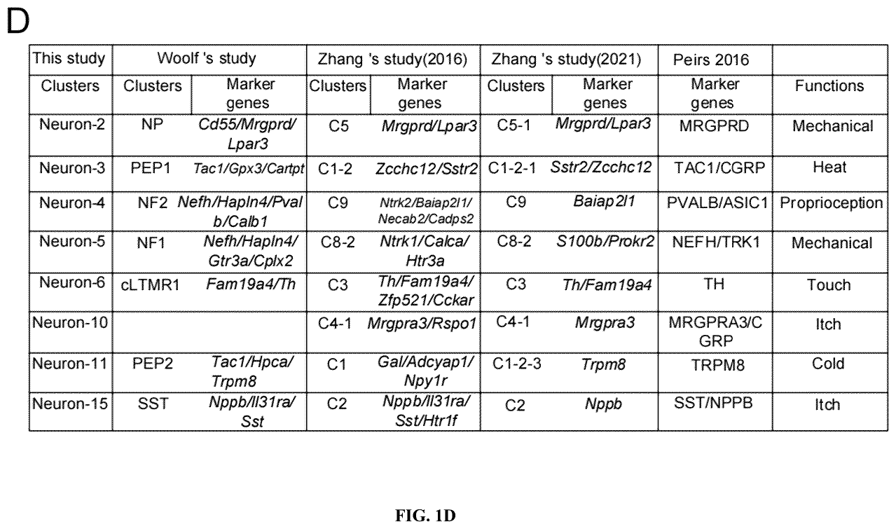

Published single-cell RNA sequencing data of mouse DRGs (Wang et al., 2021;Renthal et al., 2020; and Speir et al., 2021) were used to identify that the gene expression of different types of DRG neurons was related to their morphological and functional characteristics (Li et al., 2016). It was found that Neuron-10 highly expressed Mas-related G protein-coupled receptor A3 (MrgprA3), which might be involved in pruritus regulation (Li et al., 2016; and Peirs and P. Seal, 2016) (FIG. 1).

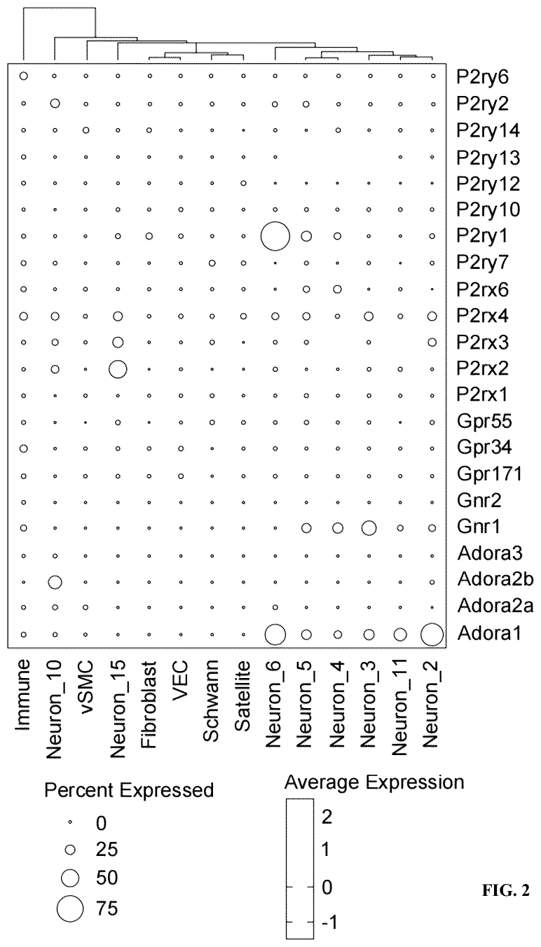

Based on previous research, the applicants analyzed the expression of genes encoding major ligand-gated ion channels. Ion channel-related genes were acquired from org.Mm.eg.db (v 3.13.0) R package, and the expression of these genes in the single-cell RNA sequencing database of mouse DRGs was detected by using the Seurat R package. Gfra1 and Adora2b were detected to be highly expressed in Neuron_10 (FIG. 2). ATP and adenosine receptors are purinergic receptors and are involved in pain regulation (Burnstock, 2016; Inoue, 2022). All Adora2b gene-encoding receptors are purinergic receptors. Notably, the above single-cell RNA sequencing data show that Neuron 10 may be involved in pruritus regulation. Therefore, the Adora2b gene is highly expressed in Neuron_10 and adenosine A2B receptor encoded thereby may also be involved in pruritus regulation.

### Example 2

### Detection of Expression of Adenosine A2B Receptor in DRG Neurons by IHC

The steps of IHC were as follows:

After a mouse underwent intraperitoneal anesthesia with pentobarbital sodium (1%), the heart was perfused with 0.1 M phosphate-buffered saline (PBS) and 4% paraformaldehyde (PFA) in sequence, followed by spinal cord separation and DRG sampling (at L4-5 segment). Separated DRG tissue was fixed with 4% PFA solution for 12 h and dehydrated with 15% and 30% sucrose solutions in sequence. After dehydration, the DRG tissue was edembedded with OCT, frozen and sectioned to a section thickness of 15 μm. A section was adhered to a slide. Later, the DRG slide was cleaned with PBS thrice, each for 10 min. Next, the DRG slide was incubated in a well-prepared block buffer (5% goat serum and 0.5% Triton X-100 in PBS) at room temperature for 1 h. Subsequently, primary antibodies (rabbit anti-A2B, 1:100; mouse anti-GFRα-1, 1:100) were applied, followed by incubation at 4° C. overnight. The antibody diluent was 2.5% goat serum and 0.05% Triton X-100 in PBS. The following day, the slide was rinsed with PBS five times, each for 10 min. The corresponding fluorescent secondary antibodies (donkey anti-mouse and donkey anti-rabbit, both 1:500) were selected according to the species of the primary antibody. Subsequently, the slide was put in a dark box to keep it away from light and incubated at room temperature for 2 h. After incubation with secondary antibodies, the slide was cleaned with PBS thrice, each for 10 min. After drying in the dark, the slide was mounted with an anti-fade mounting medium with 4′,6-diamidino-2phenylindole (DAPI). After the mounting medium was solidified, the fluorescence reaction of the DRG slide was observed and photographed under a confocal microscope.

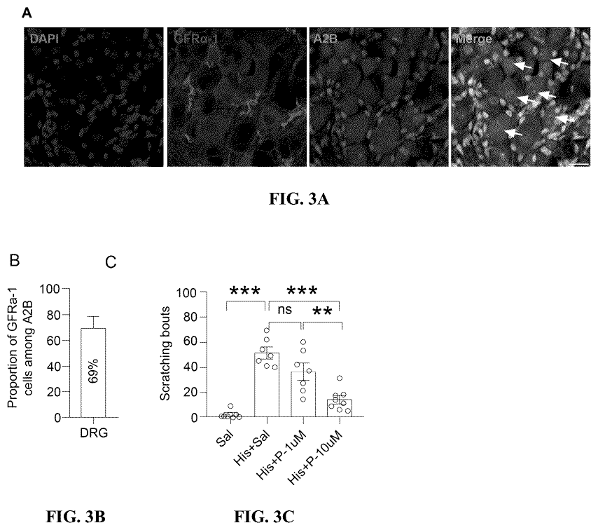

Results are shown in FIG. 3. The adenosine A2B receptor and GFRα-1 protein are co-expressed in DRG neurons (FIG. 3A), and approximately 69% of A2B-positive neurons overlap with GFRα-1 (FIG. 3B).

### Example 3

### Scratching Behavioral Experiment Investigated Whether the Adenosine A2B Receptor in the DRG was Involved in Pruritus Regulation

Refer to the prior art (Jang et al., 2015) for scratching behavior. The specific operation method was as follows: each mouse neck hair was shaved two days before the experiment, and the mouse was housed in a transparent organic plastic chamber to receive acclimatization training for two consecutive days, at least 1 h a day. Adjacent transparent plastic chambers were separated by a piece of white paper to eliminate mutual interference between mice. On the day of the experiment, histamine (250 μg/50 μL) was subcutaneously injected into the mouse neck and the mice were immediately placed in the transparent plastic chamber to record scratching behaviors within 30 min using a camera. One effective scratch was defined as follows: a mouse lifted its hind leg to scratch the skin at the histamine injection site and put the hind leg down, and despite the number of scratches in the interval between lifting the hind leg and putting it down, an effective scratching behavior was considered. To evaluate the antipruritic effect of PSB-603, the mice received an intrathecal injection of PSB-603 (the administration dose was 10 μL, and the dosing concentration was 1 or 10 μM) within 30 min before histamine injection.

Results show that epidermal injection of histamine into normal animals can effectively induce mouse scratching behaviors. Using a pharmacological method, the adenosine A2B receptor activity is blocked by intrathecal injection of selective adenosine A2B receptor antagonist PSB-603. Administration of PSB-603 in advance can effectively prevent histamine-induced scratching behaviors. Moreover, alleviating the effect of PSB-603 on scratch is related to the dose. PSB-603 at a dose of 10 μM reduces the number of histamine-induced scratches, but does not at a dose of 1 M (FIG. 3C). These results show that the adenosine A2B receptor is involved in histamine-induced pruritus. Therefore, inhibition of the adenosine A2B receptor is an effective way to treat pruritus.

### Example 4

### Scratching Behavioral Experiment Investigated Whether the Adenosine A2B Receptor in the DRG was Involved in Pruritus Regulation

Refer to the prior art (Jang et al., 2015) for scratching behavior. The specific operation method was as follows: each mouse neck hair was shaved two days before the experiment, and the mouse was housed in a transparent organic plastic chamber to receive acclimatization training for two consecutive days, at least 1 h a day. Adjacent transparent plastic chambers were separated by a piece of white paper to eliminate mutual interference between mice. On the day of the experiment, chloroquine (200 μg/50 μL) was subcutaneously injected into the mouse neck and the mice were immediately placed in the transparent plastic chamber to record scratching behaviors within 30 min using a camera. One effective scratch was defined as follows: a mouse lifted its hind leg to scratch the skin at the histamine injection site and put the hind leg down, and despite the number of scratches in the interval between lifting the hind leg and putting it down, an effective scratching behavior was considered. To evaluate the antipruritic effect of PSB-603, the mice received an intrathecal injection of PSB-603 (the administration dose was 10 μL, and the dosing concentration was 1, 10 or 20 μM) within 30 min before chloroquine injection.

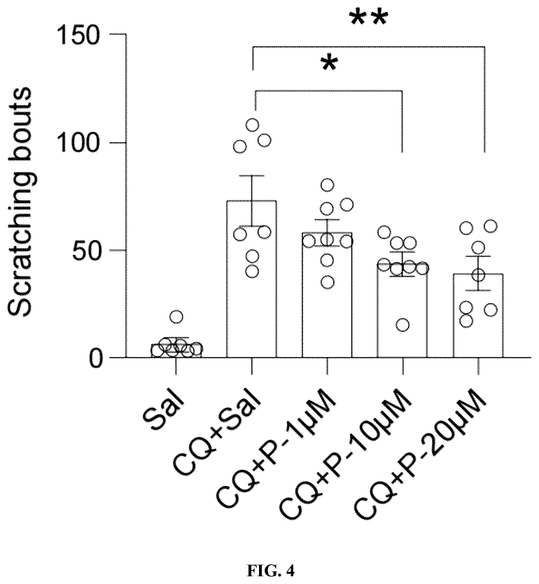

Results show that epidermal injection of chloroquine into normal animals can effectively induce mouse scratching behaviors. Using a pharmacological method, the adenosine A2B receptor activity is blocked by intrathecal injection of selective adenosine A2B receptor antagonist PSB-603. Administration of PSB-603 in advance can effectively prevent chloroquine-induced scratching behaviors. Moreover, alleviating the effect of PSB-603 on scratch is related to the dose. PSB-603 at doses of 10 and 20 μM reduces the number of chloroquine-induced scratches but does not at a dose of 1 μM (FIG. 4). These results show that adenosine A2B receptor is involved in chloroquine-induced pruritus. Therefore, inhibition of the adenosine A2B receptor is an effective way to treat pruritus.

### REFERENCES

Burnstock, G. (2016). Purinergic Mechanisms and Pain. Adv. Pharmacol. 75, 91-137. doi: 10.1016/bs.apha.2015.09.001.
Inoue, K. (2022). The Role of ATP Receptors in Pain Signaling. Neurochem. Res. 47, 2454-2468. doi: 10.1007/s11064-021-03516-6.
Jang, Y., Lee, W.-J., Hong, G.-S., and Shim, W.-S. (2015). Red ginseng extract blocks histamine-dependent itch by inhibition of H1R/TRPV1 pathway in sensory neurons. J. Ginseng Res. 39, 257-264. doi: 10.1016/j.jgr.2015.01.004
Li, C. L., Li, K. C., Wu, D., Chen, Y., Luo, H., Zhao, J. R., et al. (2016). Somatosensory neuron types identified by high-coverage single-cell RNA-sequencing and functional heterogeneity. Cell Res. 26. doi: 10.1038/cr.2015.149.
Feramisco, J. D., T. G. Berger, and M. Steinhoff (2010). Innovative management of pruritus. Dermatol Clin. 28(3): p. 467-78.Peirs, C., and P. Seal, R. (2016). Neural circuits of pain: Recent advances and current perspectives. Science (80-.). 354, 578-583.
Renthal, W., Tochitsky, I., Yang, L., Cheng, Y.-C., Li, E., Kawaguchi, R., et al. (2020).
Transcriptional Reprogramming of Distinct Peripheral Sensory Neuron Subtypes after Axonal Injury. Neuron 108, 128-144.e9. doi: 10.1016/j.neuron.2020.07.026.
Speir, M. L., Bhaduri, A., Markov, N. S., Moreno, P., Nowakowski, T. J., Papatheodorou, I., et al. (2021). UCSC Cell Browser: Visualize Your Single-Cell Data. Bioinformatics 37, 4578-4580.doi: 10.1093/bioinformatics/btab503.
Wang, K., Wang, S., Chen, Y., Wu, D., Hu, X., Lu, Y., et al. (2021). Single-cell transcriptomic analysis of somatosensory neurons uncovers temporal development of neuropathic pain. Cell Res. 31. doi: 10.1038/s41422-021-00479-9.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for screening, developing or preparing drugs for prevention and/or treatment of pruritus, comprising using an adenosine A2B receptor as a target.

2. The method according to claim 1, wherein the pruritus comprises at least one selected from the group consisting of uremic pruritus, chronic pruritus, and acute pruritus.

3. The method according to claim 1, wherein the pruritus is histamine- or chloroquine-induced pruritus.

4. The method according to claim 1, wherein the drugs comprises a component for inhibiting adenosine A2B receptor activity.

5. A reagent for prevention and/or treatment of pruritus, wherein the reagent for prevention and/or treatment of pruritus is capable of inhibiting biological functions of an adenosine A2B receptor.

6. The reagent according to claim 5, wherein the reagent for inhibiting biological functions of an adenosine A2B comprises a monoclonal or polyclonal antibody of the adenosine A2B receptor, an antagonist of the adenosine A2B receptor, and a competitive ligand of the adenosine A2B receptor.

7. The reagent according to claim 6, wherein the antagonist of the adenosine A2B receptor comprises PSB-603.

8. The reagent according to claim 7, wherein the PSB-603 is administered at a concentration of at least 10 μM.

9. The reagent according to claim 5, wherein the pruritus comprises at least one selected from the group consisting of uremic pruritus, chronic pruritus, and acute pruritus.

10. The reagent according to claim 5, wherein the pruritus is histamine- or chloroquine-induced pruritus.

11. A method for prevention and/or treatment of pruritus, wherein the method comprises administering a reagent for inhibiting biological functions of an adenosine A2B receptor to a subject in need thereof.

12. The method according to claim 11, wherein the reagent for inhibiting biological functions of an adenosine A2B comprises a monoclonal or polyclonal antibody of the adenosine A2B receptor, an antagonist of the adenosine A2B receptor, and a competitive ligand of the adenosine A2B receptor.

13. The method according to claim 12, wherein the antagonist of the adenosine A2B receptor comprises PSB-603.

14. The method according to claim 13, wherein the PSB-603 is administered at a concentration of at least 10 μM.

15. The method according to claim 11, wherein the pruritus comprises at least one selected from the group consisting of uremic pruritus, chronic pruritus, and acute pruritus.

16. The method according to claim 11, wherein the pruritus is histamine- or chloroquine-induced pruritus.
